(19) United States
(12) Patent Application Publication
(10) Pub. No.: US 2016/0331899 A1
(43) Pub. Date: Nov. 17, 2016

(54) SYRINGE ACTUATOR SYSTEM FOR DELIVERING TOXIC MEDICAL COMPOSITIONS

(71) Applicant: Alps South, LLC, St. Petersburg, FL (US)

(72) Inventor: Aldo A. Laghi, Clearwater, FL (US)

(73) Assignee: Alps South, LLC, St. Petersburg, FL (US)

(21) Appl. No.: 15/153,895

(22) Filed: May 13, 2016

Related U.S. Application Data
(60) Provisional application No. 62/173,177, filed on Jun. 9, 2015.
(60) Provisional application No. 62/161,124, filed on May 13, 2015.

(51) Int. Cl. A61M 5/20; A61M 5/315

(52) U.S. Cl. CPC A61M 5/20; A61M 5/31576; A61M 2005/2026; A61M 2202/09; A61M 2205/50; A61M 2205/6072; A61M 2205/6054

(57) ABSTRACT

A system for dispensing Botox, anti-venom, local chemotherapy, anesthetics, aesthetic fillers, endogen materials, fat for aesthetic surgery, stem cells, and lethal injection compounds though a sterile syringe. They system uses pressure, generated by a pump unit, to apply force on a syringe containing the materials to be delivered into the patient's body. The volume rate and volume per injection are adjustable through user inputs. The injection occurs when the physician activates a momentary switch. The system provides precise control of the injected material thus minimizing the inaccuracies associated with manually injected materials. Additionally, the simplicity of the system eases cleaning and sterilization practices thus minimizing risks of transferring undesirable transmissible agents.

### CROSS-REFERENCE TO RELATED APPLICATIONS

This application claims the benefit of provisional application Nos. 62/173,177, filed Jun. 9, 2015 and 62/161,124, filed May 13, 2015, the disclosures of which are each hereby incorporated by reference herein.

### BACKGROUND OF THE INVENTION

1. Field of the Invention

This invention relates to medical syringes for toxic injections.

2. Description of the Background Art

Presently, various medical procedures exist that require the delivery of a material into the dermis, subcutaneous, and muscular regions of a patient's body. While other procedures may have synonymous objectives, and therefore challenges, the scope of this invention pertains to Botox, anti-venom, local chemotherapy, anesthetics, aesthetic fillers, endogen materials, fat for aesthetic surgery, stem cells, and lethal injection compounds.

Most injections are accomplished when a physician squeezes a syringe between the palm of their hand and two fingers. The material enclosed in the syringe is then compressed and flows through a cannula needle. The needle size general ranges from 30 gauge to 16 gauge. Due to the small cross-sectional area of the needle, high pressures are required to expel the material into the injection site. Due to the density of the tissues at the injection site, additional pressure is needed to expel the fluid. The current process requires a high degree of dexterity, strength, and stamina to complete a procedure.

A syringe may be loaded with an injectable material, such as Botox, anti-venom, local chemotherapy, anesthetics, aesthetic fillers, endogen materials, fat for aesthetic surgery, stem cells, and lethal injection compounds, and then injected though a cannula into the target area. During the process, force is repeatedly placed on the syringe plunger to place the correct amount of material into desired locations. When withdrawn from the site the cannula needle leaves a void which then fills with the injected material. The amount of injected material that fills the void is determined by the force placed on the syringe plunger by the physician which, is largely determined by the dexterity of the physician.

Other delivery systems available are mechanically driven by the operator and utilize mechanical advantage to ease the injection process. The manual pumping of these systems make it difficult for the operator to maintain accuracy of the injection site. During the squeezing process the tendency is for the operator to drift the needle up, down, or side-to-side. Additionally, because of the moving mechanical parts, these systems present sterilization concerns. The only method to adequately clean and sterilize require the system to be to completely disassembled, a time consuming task.

Also available in the medical industry are a number of syringe actuators that are driven electrically, pneumatically, manually, hydraulically or peristaltically. Some prior art examples are disclosed in US 2015/0038906 A1, U.S. Pat. No. 6,957,747 B2 and U.S. Pat. No. 8,123,718 B2. However, they all present one or more of the following disadvantages:
The rate of injection is either not adjustable or controllable to a preset value.
The rate of injection is not constant during the procedure. This is a problem common to pneumatic, peristaltic and manual injectors. The Friction between the rubber seal of the syringe plunger and the inside of the syringe barrel, change from static to dynamic at the start or re-start of the injection. It is well known that the coefficient of static friction between two solids is considerably higher than the coefficient of dynamic friction. This result in a jerking motion of the plunger as it is pushed forward. This problem exists even if the force applied on the plunger is maintained constant.
They are bulky, particularly the electrically driven actuators, and therefore do not permit fine control of the needle pressure nor the location of the needle in the patient's anatomy.
They do not prevent the injection of the wrong medication into the patient.
They do not maintain a record of the medication, patient, date, rate of injection, or total injection amount.
The present invention syringe actuator is designed to overcome all the above disadvantages of the prior art actuators.

The present invention comprises an instrument may be utilized by a physician as an alternative to manually pumping a syringe. The present invention syringe actuator system is nimble in the hand of the care giver, thus allowing fine control of the location in the patient anatomy. This is particularly advantageous when the substance injected must be deposited in multiple locations along a preset path in the anatomy, or when a bead of the substance is wanted to be applied to the anatomy of the patient. Such as is the case with Botox, Derma fillers, fat, other aesthetic surgery substances.

Therefore, it is an object of this invention to provide an improvement which overcomes the aforementioned inadequacies of the prior art devices and provides an improvement which is a significant contribution to the advancement of the medical injection art.

Another object of this invention is to provide a syringe actuator system to preset and control the amount of substance to be injected. Such control allows for a preset constant rate of injection, or a preset variable rate of injection.

Another object of this invention is to provide a syringe actuator system for data collection of injections to be stored with a patient's medical records.

Another object of this invention is to provide a syringe actuator system allowing the caregiver to provide evidence based care for purposes of liability insurance, reimbursement rates, liability defense.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention comprises a syringe actuator system which basically includes a dispensing unit (100) connected to a remote control unit (300) via a flexible motion transfer member (200). The dispensing unit (100) is comprised of a hand piece adapted to have a syringe removably mounted therein. Such syringes are conventional and include a cylinder for retaining an injectable material therein, a hypodermic needle mounted at one end thereof and a plunger mounted within the cylinder at the opposite end for ejecting the material from the cylinder through the needle. The hand piece also includes a syringe actuator connected to the syringe plunger by a transfer bar mechanism. The syringe actuator is connected to the remote control unit by a flexible motion transfer member (200) which can be a flexible hydraulic tube, a Bowden cable, a rotatable flexible cable actuator, an electroactive polymer actuator or a thermo bimorph actuator. The remote control unit (300) includes a driving mechanism for transferring either rotating motion or linear motion through the flexible motion transfer member (200) to the transfer bar mechanism in the hand piece to apply a force to the syringe plunger to inject the proper amount of material into the injection site. The instrument may be controlled by the physician to inject material when the physician activates an inject switch on the hand piece. Furthermore, the flow rate of the injection and/or amount of injection material may be predetermined by the physician via a main control unit on the remote control unit. The hand piece is designed as a light weight injection gun adapted to hold the injection syringe which is easily maneuverable by the physician thus allowing a clear line of site along the needle for accurate positioning. This enhances the ergonomics of the instrument minimizing hand fatigue, which can result in improper positioning of the needle at the injection site.

Sterilization requirements for the injection system of the present invention are simple as are described hereinafter.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 shows a pictorial view of a preferred embodiment of the present invention.

FIG. 2A shows a plan view of a preferred embodiment of the dispensing unit with the syringe in its fully dispensed position.

FIG. 2B shows a plan view of a preferred embodiment of the dispensing unit with the syringe in its fully retracted position.

FIG. 3 is a pictorial view of a preferred embodiment of the remote control unit.

Similar reference characters refer to similar parts throughout the several views of the drawings.

### DETAILED DESCRIPTION OF THE PREFERRED EMBODIMENT

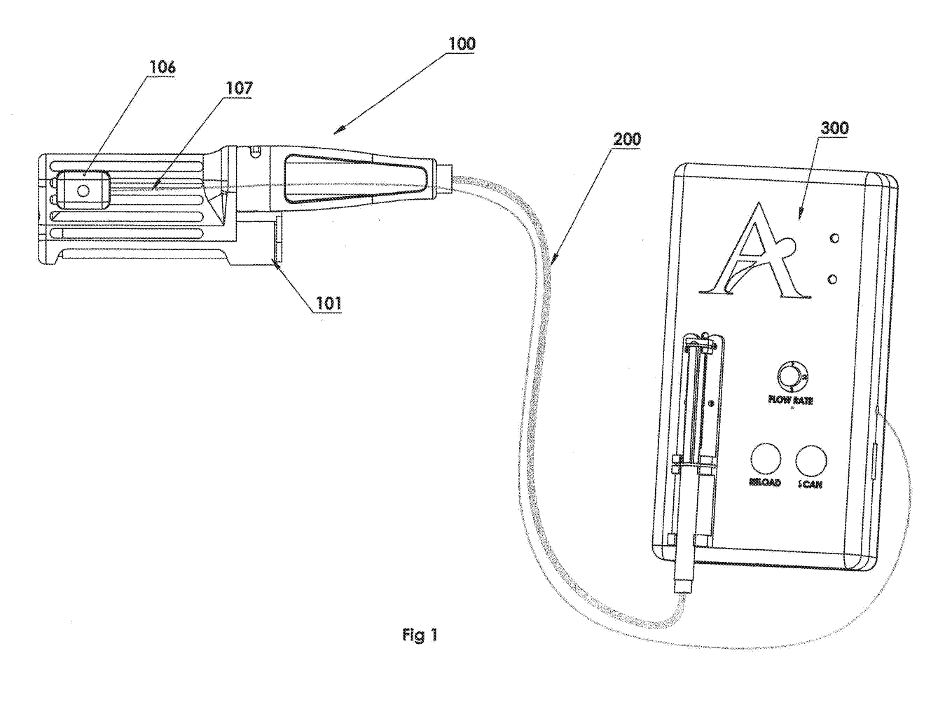

Referring to FIG. 1, a preferred embodiment of the syringe actuator system of the present invention includes a dispensing unit (100) connected to a remote control unit (300) via a flexible motion transfer member (200).

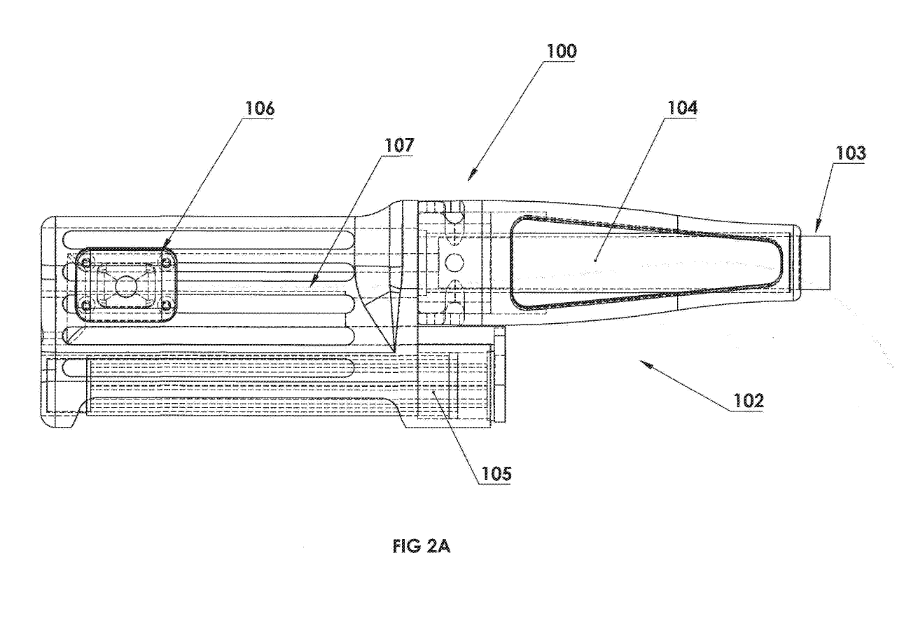

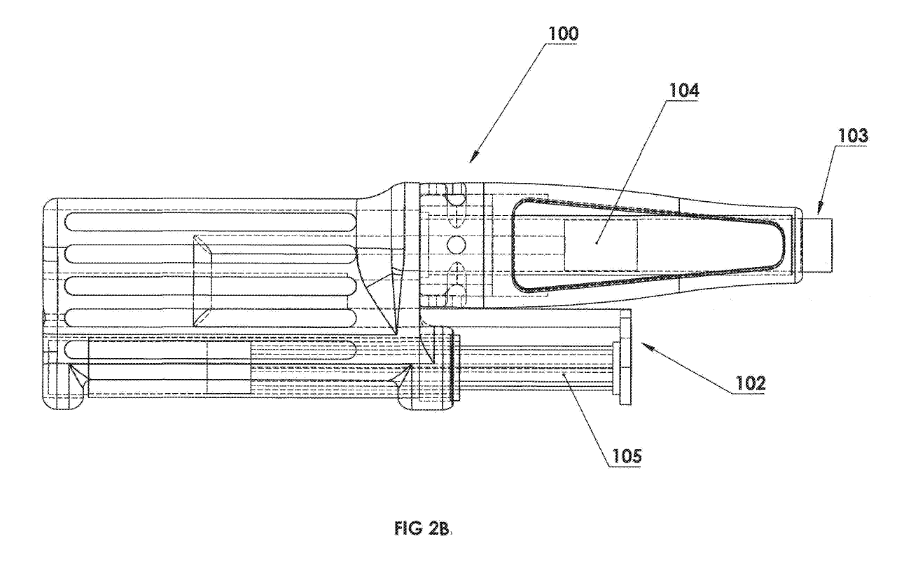

As illustrated in FIG. 2A, the dispensing unit (100) is designed as a light weight, ergonomically friendly hand piece adapted to have a syringe (105) removably mounted therein. The hand piece is designed as a light weight injection gun adapted to hold the injection syringe which is easily maneuverable by the physician thus allowing a clear line of site along the needle for accurate positioning. This enhances the ergonomics of the instrument minimizing hand fatigue, which can result in improper positioning of the needle at the injection site. Such injection syringes are conventional and include a cylinder for retaining an injectable material therein, a hypodermic needle (not shown) mounted at one end thereof and a plunger mounted within the cylinder at the opposite end for ejecting the material from the cylinder through the needle. In FIG. 2A, the plunger is shown in its fully dispensed position.

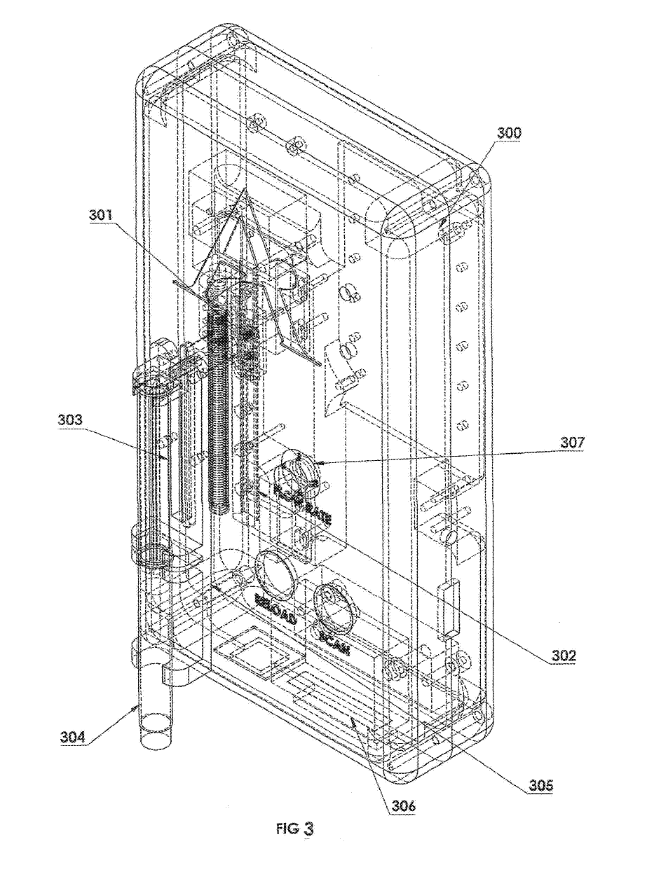

In FIG. 2B, the plunger is shown in its full retracted position. As best illustrated in FIG. 2B, the hand piece also includes a syringe actuator is connected to the syringe plunger by a transfer bar mechanism (102). In this preferred embodiment, the syringe actuator further includes hydraulic cylinder (103) and piston (104) connected to the transfer bar mechanism (102). The hydraulic cylinder (103) is fluidically connected to a hydraulic pumping unit in the remote control unit (300) shown in FIG. 3 via a flexible hydraulic tube which has an inner diameter is between 0.050 and 0.125 inches and a length between 3 and 8 feet long. Referring again to FIG. 2A, the hand piece further includes switch (106) for manual control by the user for activating the driving mechanism located in the remote control unit (300). The switch (106) is connected to the driving mechanism via switch cable (107).

As illustrated in FIG. 3, the remote control unit (300) includes a driving mechanism for transferring either rotating motion or linear motion through the flexible motion transfer member (200) to the transfer bar mechanism (102) in the hand piece to apply a force to the syringe plunger to inject the proper amount of material into the injection site. In the preferred embodiment, the driving mechanism includes hydraulic piston (303) within hydraulic cylinder (304) fluidically connected to the flexible motion transfer member (200) which, in this case, is a hydraulic tube. The hydraulic cylinder (103) connected to hydraulic cylinder (304) via a hydraulic tube are fully filled with a non-compressible fluid.

In the preferred embodiment as illustrated in FIG. 3, the hydraulic piston (303) is connected to a slide rail (302) which is linearly driven by lead screw (301). The lead screw (301) is rotated by an electric motor (not designated) the speed of which is controlled by a potentiometer (307) adjustable by the attending physician. The electric motor is controlled by the physician to inject material when the physician activates an inject switch (106) on the hand piece. Thus, the flow rate of the injection may be predetermined by the physician by adjusting the potentiometer on the remote control unit and the amount of injection material may be determined by the physician by activating the electric motor via the switch (106).

The remote control unit (300) further includes a scanner (306) connected to a microprocessor connected to the electric motor which is used to control the injection process to an individual patient. Thus, the scanner may be used to cross-check a patient's record to prevent the injection of the wrong medication, to maintain a record of the total amount injected, allows for the control of a constant preset rate of injection, allows for the control of a preset variable rate of injection, allows for collecting data to be stored with the patient record, and allows the caregiver to provide evidence of the injections performed on individuals for purposes of liability insurance, reimbursement rates and liability defense.

Sterilization requirements for the injection system may be simple. The pump unit may be remotely connected to the injection gun though disposable tubing. The injection unit has relatively few parts including a syringe containing the botulinum toxin and/or adipose tissue, a syringe which converts the hydraulic pressure from the pump unit to a force on the injection syringe, and a transfer bar. The entire system may be sterilized in an autoclave. In one variation the syringes are non-reusable, requiring the physician to use new sterile syringes each operation. In this variation the only part requiring cleaning and sterilization is the frame of the injection gun and the inject switch bladder and line. In another variation, all components are disposable except for the injection gun frame. In another variation, all components are non-reusable.

Although the preferred embodiment as illustrated in FIGS. 1, 2A, 2B and FIG. 3 are directed to a hydraulic actuating system, it is within the confines of the present invention that other actuating systems could equally be employed such as linear or rotational motor mechanisms mounted in the remote control unit (300) for driving a Bowden cable, a rotatable flexible cable actuator, an electroactive polymer actuator or a thermo bimorph actuator as the flexible motion transfer member (200).

The present invention has been described in terms of various embodiments. It will be appreciated by those skilled in the art that various changes and modifications may be made to the embodiments without departing from the spirit or scope of the invention. It is not intended that the invention be limited to the embodiments shown and described. It is intended that the invention include all foreseeable modifications to the embodiments shown and described. It is intended that the invention be limited in scope only by the claims appended hereto.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A dispensing device for injecting toxic or otherwise dangerous medical compositions subcutaneously comprising:
a sterile, autoclavable, hand piece having a sterile syringe removably mounted therein, said syringe comprising a plunger slidably mounted within a syringe barrel, said hand piece having a first driving mechanism therein connected to said syringe for actuating said syringe plunger to inject said compositions subcutaneously into a patient;
a remote control unit having a second driving mechanism mounted therein;
a connection means between said first and second driving mechanisms that transfers motion of the second driving mechanism in the remote control unit to the first driving mechanism in the hand piece without any time delay, change in displacement of the injection and change in the speed of the injection; and
said hand piece having a actuator mounted thereon fort actuating said second driving mechanism in said remote control unit.

2. A dispensing device as claimed in claim 1, further comprising;
said remote control unit having a reader (bar code, qr, rfid) that detects the serial number of the syringe to be mounted in the hand piece;
a microprocessor connected to the remote control unit wirelessly or electrically wired having software or an algorithm stored therein that allows the operation of the remote control unit after the syringe is verified;
a data storage system local or remote to said dispensing device for maintaining records of any or combination of the following: patient data, medication used, injection rate and amount and date of injections.

3. A dispensing device as claimed in claim 1, further comprising;
said first and second driving mechanisms comprise hydraulic piston/cylinder assemblies connected by a flexible hydraulic tube.

4. A dispensing device as claimed in claim 3, wherein said flexible tube has an outside diameter of between 1/32 and ⅛ of an inch and a length between 2-12 feet, said hydraulic cylinders and said flexible tube being is filled with an incompressible liquid thereby providing an incompressible medium to deliver controlled thrust from the piston of the second driving mechanism to the piston of the first driving mechanism to the plunger of the syringe located in the hand unit.

5. A dispensing device as claimed in claim 1, wherein said connection means is a Bowden cable.

6. A dispensing device as claimed in claim 1, wherein said connection means is a Bowden cable.

7. A dispensing device as claimed in claim 3, wherein said second driving mechanism further comprises a DC electric rotary motor and a potentiometer for controlling the speed of the motor and a linear screw drive that converts the rotary motion of the electric rotary motor to linear motion to drive the piston of the piston/cylinder assembly of the second driving mechanism to drive the non-compressible liquid in the hydraulic tube and apply a force to the piston of the piston/cylinder assembly of the first driving mechanism cylinder to drive the plunger of the syringe in the hand piece.

8. A dispensing device as claimed in claim 3, wherein said the hydraulic piston-cylinder assembly located in the hand piece is also a sterile syringe.

9. A dispensing device as claimed in claim 3, wherein said the hydraulic tube is a sterile medical tube.

10. A dispensing device as claimed in claim 3, wherein said hydraulic piston-cylinder assembly located in the remote control unit is also a sterile syringe.

11. A dispensing device as claimed in claim 7, wherein said actuator comprises a momentary electric switch which is wirelessly or wired to said DC electric motor in said remote control unit.

12. A dispensing device as claimed in claim 1, wherein said injection compounds include any one of the following: Botox, anti-venom, local chemotherapy, anesthetics, aesthetic fillers, endogen materials, fat for aesthetic surgery, stem cells, lethal injection compounds.

13. A dispensing device as claimed in claim 1, wherein the connection means is any one of the following:
a. rotating flexible cable actuator,
b. electroactive polymer actuator,
c. thermo bimorph actuator.

14. A dispensing device as claimed in claim 11, wherein said momentary electric switch is easily removably clipped to the hand piece covered by a disposable sterile sheath.
